(19) United States
(12) Patent Application Publication
(10) Pub. No.: US 2007/0148689 A1
(43) Pub. Date: Jun. 28, 2007

(54) Compositions and methods for the prevention, treatment and detection of tuberculosis and other diseases

(76) Inventor: Kathryn Leishman, Brooklyn, NY (US)

Correspondence Address: NOVAK DRUCE & QUIGG, LLP, 1300 EYE STREET NW, SUITE 1000 WEST TOWER, WASHINGTON, DC, 20005, US

(21) Appl. No.: 11/703,796

(22) Filed: Feb. 8, 2007

Related U.S. Application Data
(63) Continuation-in-part of application No. 10/265,190, filed on Oct. 7, 2002.
Application No. 10/265,190 is a continuation-in-part of application No. 10/018,243, now abandoned.
(60) Provisional application No. 60/206,518, filed on May 22, 2000.
(60) Provisional application No. 60/194,766, filed on Apr. 3, 2000.

(51) Int. Cl. C12Q 1/70; C12Q 1/68

(52) U.S. Cl. 435/6; 435/5

(57) ABSTRACT

Methods and compositions are provided for the prevention and treatment of infectious diseases such as syphilis, tuberculosis, pneumonia, other bacterial infections, AIDS, and other viral infections. Many of the compositions are active against carbon monoxide dehydrogenase (“CODH”), and include substances such as antigens, antibodies specific for CODH, and other inhibitors of CODH such as nickel and molybdenum metal chelators. The methods and compositions are particularly suited for treatment of diseases from previously under recognized anaerobic or facultative anaerobic pathogens such as Mycobacterium tuberculosis and Mycobacterium pneumonia.

This application is a continuation-in-part of U.S. Ser. No. 10/265,190, which is a continuation-in-part of U.S. Ser. No. 10/018,243, filed Dec. 18, 2001, which is a continuation of international application no. PCT/US00/16679, filed Jun. 19, 2000, which receives priority from provisional application 60/206,518 filed May 22, 2000 and 60/194,766 filed Apr. 3, 2000. All prior applications are incorporated by reference in their entireties.

### FIELD OF THE INVENTION

This invention relates to compositions and methods for detecting, preventing and treating infectious diseases such as Mycobacterium tuberculosis (“M. TB”), M. pneumonia (“M. TP”), and to new classes of antibiotics effective against anaerobic and facultative anaerobic microorganisms.

### BACKGROUND OF THE INVENTION

Treatment and prophylaxis of infectious diseases have been advanced tremendously by the discovery of antibiotics and vaccines. The discovery and implementation of antibiotics to kill bacteria has greatly increased human life span and the discovery of the role of the immune system in warding off and reversing viral disease has been exploited to great benefit by vaccination programs against those diseases. Despite those great successes, however, new modalities of action for antibiotics against the bacteria are needed in view of the development of resistance to those same antibiotics. At the same time, mankind's creativity and understanding of the molecular biology behind disease is challenged anew by the AIDS crisis. Despite almost two decades of intensive research there is still no cure for AIDS, though it appears that effective treatment for various infections, including HIV, that 30%, afflict AIDS patients prolongs their lives. Thus, modern society is faced with two major challenges: the prevention, treatment and detection of intractable disease such as tuberculosis, syphilis, and AIDS and the development of antibiotics that utilize new molecular modalities against bacteria that resist the old treatments.

The problems of the rapidly growing global incidence of tuberculosis and microbial resistance have been often described by many workers in the healthcare industry and are well known to skilled artisans in that field. In fact, virtually everyone understands the need for new materials and methods for combating these problems. Because its pathomechanism is still not understood, any new information regarding how tuberculosis develops could clearly be used in many different ways to improve diagnosis, therapy and treatment of that disease. This disease is a global threat, but its incidence is especially common in late-staging AIDS patients, a majority of whom suffer from it. Likewise, any new information about structures of unique bacterial components such as bacterial enzymes which catalyze reactions necessary for the bacteria clearly could be used, for example, by techniques variously termed “rational drug design” that exploit such information to predict what structures can work as antibiotics.

### SUMMARY OF THE INVENTION

One embodiment is a method for the prevention of an infectious disease in a human subject, comprising the steps of providing a pharmaceutical composition comprising at least one substance selected from the group consisting of CODH antigen, Anti-CODH antibody, CODH nucleotide, CODH antisense nucleic acid, CODH inhibitor, and CODH chelator; and administering the composition to the patient in a form that allows uptake by cells of the subject.

Another embodiment of the invention is a method for the treatment of an infectious disease in a human subject, comprising the steps of providing a pharmaceutical composition comprising at least one substance selected from the group consisting of CODH antigen, Anti-CODH antibody, CODH nucleotide, CODH antisense nucleic acid, CODH inhibitor, and CODH chelator; and administering the composition to the patient in an form that allows uptake by cells of the subject.

In yet another embodiment the invention is a pharmaceutical composition for the prevention of an infectious disease in a human subject, comprising at least one substance selected from the group consisting of CODH antigen, Anti-CODH antibody, CODH nucleotide, CODH antisense nucleic acid, CODH inhibitor, and CODH chelator.

Yet another embodiment provides compositions and methods for inhibiting Mycobacterium tuberculosis and/or Mycobacterium pneumonia via the use of copper chelators. One embodiment inhibits bacteria via copper chelation by addition of a copper chelate to a physiological space such as by injection into the vascular space or by oral ingestion. Another embodiment is a method for alleviating the effect of disease associated with bacteria such as Mycobacterium tuberculosis and Mycobacterium pneumonia by: 1) detecting a positive sign of bacteria infection; and 2) responding to the detected positive sign of infection via administration of a copper chelator or other agent that inhibits CODH. In an embodiment the copper chelator is triethylenetetramine dihydrochloride (trientine dihydrochloride).

Other embodiments will be readily appreciated from reading the specification.

The inventor discovered that M. tuberculosis should contain an enzyme—carbon monoxide dehydrogenase—which, under various circumstances, could affect health by producing carbon monoxide, nitric oxide, carbon dioxide, and/or nitrous oxide. In particular, the inventor determined that the effects of these toxic compounds account for many of the heretofore unexplained symptoms of tuberculosis. To follow up this insight, the inventor conducted Southern blots in 1997, but obtained inconclusive data. However, when the genome of M. tuberculosis was published, it was found that, the genome, indeed, contained carbon monoxide dehydrogenase. According to this embodiment of the invention, inhibiting this enzyme by, for example exposure to inhibitors, including chemical inhibitor(s) or antibodies, disables the mycobacterium and alleviates problems of this scourge. Furthermore, the inventor revealed that this enzyme is involved in producing symptoms of Lyme disease and that blood serum from the western fence lizard has one or more binding factors that, in an analogous way, inhibit lyme disease in that animal.

In M. TB. phosphotransacetylase and acetate kinase often are found in the presence of CODH, yet are far more prevalent than CODH. M. TB contains all three enzymes, whereas other organisms such as T. pallidum indicates that this other organism contains phosphotransacetylase and acetate kinase, but not CODH. Moreover, because acetate kinase and phosphotransacetylase are critical to the metabolism of the microorganisms in which they are found, the inventor further realized that inhibiting these two other enzymes or disabling CODH, in the presence of CODH or independent of it, presents a novel way of disabling CODH or, beyond that, the entire microorganism, thereby treating the related disease.

Based on these insights, preferred embodiments of the invention utilize knowledge of the structure(s) of one or more of the enzymes, inhibitors of the enzymes, and/or methods for inhibition of the enzymes to prevent and/or treat disease. Most desirably, knowledge of the structure of each enzyme permits the development of new and more efficacious medical compositions and methods for inhibiting the enzyme, as well as methods for creating antibodies directed against the enzymes—thereby preventing the growth and proliferation of TB and other microorganisms. The strategy of determining the structure of such enzyme and then deriving or finding inhibitors of the enzyme represents a unique departure from the concept of traditional antibiotic treatment.

The compositions and methods are outlined and described in further detail in section B: “Implementation”.

Based on this insight monoclonal antibodies directed against all subunits of CODH, were prepared which are useful for the detection, treatment and prevention of M. tuberculosis and other diseases related to anaerobic microorganisms in which CODH might figure. These new medical agents inhibit their target microorganisms and represent a new class of medically active substances hereinafter termed “Microbe Inhibiting Agent.” Although the initial discovery was pertained to CODH, the invention also features agents that interfere with other enzymes needed for an aerobic and cumulative anaerobic organism such as M. tuberculosis.

Implementation

The discovery relates to compositions and methods for detection, prophylaxis, and treatment of TB and other disease states that arise from activity of CODH. Such other disease states include, for example, symptoms of AIDS. In fact, screen tests for TB often are not reliable in immune-suppressed patients (AIDS patients and others) and some symptoms attributed to “AIDS” arise from undetected TB, whose symptoms mimic those of AIDS. Thus one embodiment of the invention is an AIDS therapy.

In preferred embodiments, an M. TB CODH enzyme is targeted by a “Microbe Inhibiting Agent.” In a preferred embodiment, an enzyme of the organism which is not found naturally in the human is crystallized and a 3 dimensional structure is obtained. The obtained structure is used to design a “Microbe Inhibiting Agent” that is active against the organism, but substantially not active against human cells. That is, in preferred embodiments Microbe Inhibiting Agents are prepared and used that act by interfering with a target Enzyme of a microorganism. The microorganism may be anaerobic or facultative anaerobic, and preferably is the M. TB organism.

In another preferred embodiment the Microbe Inhibiting Agent is a molecule that is obtained from or modeled after a Lyme disease inhibiting substance, and preferably an antibody, from the western fence lizard.

Compositions and Methods that Employ an “Enzyme” According to the Present Invention

One embodiment of the invention is a method that uses at least one protein of the M. TB (or related) organism directly or indirectly (to make antibody) to derive an anti-microbial having an inhibiting or killing activity against at least one anaerobic or facultative anaerobic bacteria. Preferred embodiments that relate to all such proteins are described next.

“Enzyme” refers in its broadest sense to any enzyme made by TB or a related organism that is distinguished from a corresponding enzyme in humans. In most embodiments, “Enzyme” refers to carbon monoxide dehydrogenase of an organism, for example, M. TB. In the preferred embodiment where antibodies are made against the enzyme, immunologically cross-reactive peptides and proteins are included within this meaning. Preferably the enzyme is carbon monoxide dehydrogenase.

“Enzyme homolog” refers to a homolog, particularly of CODH enzyme but optionally one of the other enzymes, naturally occurring enzyme, or active fragments thereof, which are encoded by mRNAs transcribed from cDNA coding for the enzyme.

“Active” refers to those forms of Enzyme which retain biologic and/or immunologic activities of any naturally occurring Enzyme.

“Naturally occurring Enzyme” refers to Enzyme produced by human cells that have not been genetically engineered and specifically contemplates various Enzymes arising from post-translational modifications of the polypeptide including but not limited to acetylation, carboxylation, glycosylation, phosphorylation, lipidation and acylation.

“Derivative” refers to polypeptides derived from naturally occurring Enzyme by chemical modifications such as ubiquitination, labeling (e.g., with radionuclides, various enzymes, etc.), pegylation (derivatization with polyethylene glycol), or by insertion (or substitution by chemical synthesis) of amino acids (aa) such as ornithine, which do not normally occur in human proteins.

“Microbe Inhibiting Agent” refers to an active compound or other molecule that destroys or inhibits a bacterium by interfering with the activity of one or more Enzymes of that bacterium. The preferred Enzyme in this context is carbon monoxide dehydrogenase (“CODH”). The bacterium preferably is an anaerobic or facultative anaerobic bacterium and preferably M. tuberculosis.

“Recombinant variant” refers to any polypeptide differing from naturally occurring Enzyme by as insertions, deletions, and substitutions, created using recombinant DNA techniques. Guidance in determining which as residues may be replaced, added or deleted without abolishing activities of interest, such as cell adhesion and chemotaxis, may be found by comparing the sequence of the particular Enzyme with that of homologous molecules and minimizing the number of as sequence changes made in regions of high homology.

Preferably, as “substitutions” are the result of replacing one as with another as having similar structural and/or chemical properties, such as the replacement of a leucine with an isoleucine or valine, an aspartate with a glutamate, or a threonine with a serine, i.e., conservative as replacements. “Insertions” or “deletions” are typically in the range of about 1 to 5 aa. The variation allowed may be experimentally determined by systematically making insertions, deletions, or substitutions of as in an Enzyme molecule using recombinant DNA techniques and assaying the resulting recombinant variants for activity.

A polypeptide “fragment,” “portion,” or “segment” is a stretch of as residues of at least about 5 aa, often at least about 7 aa, typically at least about 9 to 13 aa, and, in various embodiments, at least about 17 or more aa. To be active, any Enzyme polypeptide must have sufficient length to display biologic and/or immunologic activity on their own or when conjugated to a carrier protein such as keyhole limpet hemocyanin.

An “oligonucleotide” or polynucleotide “fragment”, “portion,” or “segment” is a stretch of nucleotide residues which is long enough to use in polymerase chain reaction (PCR) or various hybridization procedures to amplify or simply reveal related parts of mRNA or DNA molecules. One or both oligonucleotide probes will comprise sequence that is identical or complementary to a portion of Enzyme where there is little or no identity or complementarity with any known or prior art molecule. The oligonucleotide probes will generally comprise between about 10 nucleotides and 50 nucleotides, and preferably between about 15 nucleotides and about 30 nucleotides.

“Activated monocytes” as used herein refers to the activated, mature monocytes or macrophages found in immunologically active tissues.

“Animal” as used herein may be defined to include human, domestic or agricultural (cats, dogs, cows, sheep, etc) or test species (mouse, rat, rabbit, etc).

“Recombinant” may also refer to a polynucleotide which encodes Enzyme and is prepared using recombinant DNA techniques. The DNAs which encode. Enzyme may also include allelic or recombinant variants and mutants thereof.

“Nucleic acid probes” are prepared based on the cDNA sequences which encode Enzyme provided by the present invention. Nucleic acid probes comprise portions of the sequence having fewer nucleotides than about 6 kb, usually fewer than about 1 kb. After appropriate testing to eliminate false positives, these probes may be used to determine whether mRNAs encoding Enzyme are present in a cell or tissue and to isolate similar nucleic acid sequences from chromosomal DNA extracted from such cells or tissues as described by Walsh P S et al (1992, PCR Methods Appl 1:241-250).

Probes may be derived from naturally occurring or recombinant single- or double-stranded nucleic acids or be chemically synthesized. They may be labeled by nick translation, Klenow fill-in reaction, PCR or other methods well known in the art. Probes of the present invention, their preparation and/or labeling are elaborated in Sambrook J et al (1989) Molecular Cloning: A Laboratory Manual, Cold Spring Harbor Laboratory, Cold Spring Harbor N.Y.; or Ausubel F M et al (1989) Current Protocols in Molecular Biology, John Wiley & Sons, New York City, both incorporated herein by reference.

Alternatively, recombinant variants encoding these same or similar polypeptides may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations may also be introduced to modify the properties of the polypeptide, including but not limited to ligand-binding affinities, interchain affinities, polypeptide degradation and turnover rate. One example involves inserting a stop codon into the nucleotide sequence to limit the size of Enzyme so as to provide a binding, non-activating ligand of smaller molecular mass which would serve to block the activity of the natural Enzyme.

As used herein, the term “transformed cell” means a cell into which (or into an ancestor of which) a nucleic acid molecule encoding a polypeptide of the invention has been introduced, by means of, for example, recombinant DNA techniques or viruses.

A “structural gene” is a DNA sequence that is transcribed into messenger RNA (mRNA) which is then translated into a sequence of amino acids characteristic of a specific polypeptide.

An “isolated DNA molecule” is a fragment of DNA that has been separated from the chromosomal or genomic DNA of an organism. Isolation also is defined to connote a degree of separation from original source or surroundings. For example, a cloned DNA molecule encoding an avidin gene is an isolated DNA molecule. Another example of an isolated DNA molecule is a chemically-synthesized DNA molecule, or enzymatically-produced cDNA, that is not integrated in the genomic DNA of an organism. Isolated DNA molecules can be subjected to procedures known in the art to remove contaminants such that the DNA molecule is considered purified, that is towards a more homogeneous state.

“Complementary DNA” (cDNA) is a single-stranded DNA molecule that, is formed from an mRNA template by the enzyme reverse transcriptase. Typically, a primer complementary to portions of the mRNA is employed for the initiation of reverse transcription. Those skilled in the art also use the term “cDNA” to refer to a double-stranded DNA molecule that comprises such a single-stranded DNA molecule and its complementary DNA strand.

The term “expression” refers to the biosynthesis of a gene product. For example, in the case of a structural gene, expression involves transcription of the structural gene into mRNA and the translation of mRNA into one or more polypeptides.

A “cloning vector” is a nucleic acid molecule, for example, a plasmid, cosmid, or bacteriophage that has the capability of replicating autonomously in a host cell. Cloning vectors typically contain (i) one or a small number of restriction endonuclease recognition sites at which foreign DNA sequences can be inserted in a determinable fashion without loss of an essential biological function of the vector, and (ii) a marker gene that is suitable for use in the identification and selection of cells transformed with the cloning vector. Marker genes include genes that provide tetracycline resistance or ampicillin resistance, for example.

An “expression vector” is a nucleic acid construct, generated recombinantly or synthetically, bearing a series of specified nucleic acid elements that enable transcription of a particular gene in a host cell. Typically, gene expression is placed under the control of certain regulatory elements, including constitutive or inducible promoters, tissue-preferred regulatory elements, and enhancers. Such a gene is said to be “operably linked to” or “operatively linked to” the regulatory elements, which means that the regulatory elements control the expression of the gene.

A “recombinant host” may be any prokaryotic or eukaryotic cell that contains either a cloning vector or expression vector. This term also includes those prokaryotic or eukaryotic cells that have been genetically engineered to contain the cloned gene(s) in the chromosome or genome of the host cell.

“Antisense RNA”: In eukaryotes, RNA polymerase II catalyzes the transcription of a structural gene to produce mRNA. A DNA molecule can be designed to contain an RNA polymerase II template in which the RNA transcript has a sequence that is complementary to that of a preferred mRNA. The RNA transcript is termed an

“antisense RNA”. Antisense RNA molecules can inhibit mRNA expression (for example, Rylova et al., Cancer Res, 62(3):801-8, 2002; Shim et al., Int J Cancer, 94(1):6-15, 2001).

“Antisense DNA or DNA decoy or decoy molecule”: With respect to a first nucleic acid molecule, a second DNA molecule or a second chimeric nucleic acid molecule that is created with a sequence, which is a complementary sequence or homologous to the complementary sequence of the first molecule or portions thereof, is referred to as the “antisense DNA or DNA decoy or decoy molecule” of the first molecule. The term “decoy molecule” also includes a nucleic molecule, which may be single or double stranded, that comprises DNA or PNA (peptide nucleic acid), and that contains a sequence of a protein binding site, preferably a binding site for a regulatory protein and more preferably a binding site for a transcription factor. Applications of antisense nucleic acid molecules, including antisense DNA and decoy DNA molecules are known in the art, for example, Morishita et al., Ann N Y Acad Sci, 947:294-301, 2001; Andratschke et al., Anticancer Res, 21:(5)3541-3550, 2001. Antisense DNA or PNA molecules can inhibit, block, or regulate function and/or expression of CODH gene.

The term “operably linked” is used to describe the connection between regulatory elements and a gene or its coding region. That is, gene expression is typically placed under the control of certain regulatory elements, including constitutive or inducible promoters, tissue-specific regulatory elements, and enhancers. Such a gene is said to be “operably-linked to” or “operatively linked to” the regulatory elements.

“Sequence homology” is used to describe the sequence relationships between two or more nucleic acids, polynucleotides, proteins, or polypeptides, and is understood in the context of and in conjunction with the terms including: (a) reference sequence, (b) comparison window, (c) sequence identity, (d) percentage of sequence identity, and (e) substantial identity or “homologous.”

(a) A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and most preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and most preferably about 100 nucleotides or about 300 nucleotides.

(b) A “comparison window” includes reference to a contiguous and specified segment of a polynucleotide sequence, wherein the polynucleotide sequence may be compared to a reference sequence and wherein the portion of the polynucleotide sequence in the comparison window may comprise additions, substitutions, or deletions (i.e., gaps) compared to the reference sequence (which does not comprise additions, substitutions, or deletions) for optimal alignment of the two sequences. Generally, the comparison window is at least 20 contiguous nucleotides in length, and optionally can be 30, 40, 50, 100, or longer. Those of skill in the art understand that to avoid a misleadingly high similarity to a reference sequence due to inclusion of gaps in the polynucleotide sequence a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 8: 2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wis., USA; the CLUSTAL program is well described by Higgins and Sharp, Gene, 73: 237-244, 1988; Higgins and Sharp, CABIOS:11-13, 1989; Corpet, et al., Nucleic Acids Research, 16:881-90, 1988; Huang, et al., Computer Applications in the Biosciences 8:1-6, 1992; and Pearson, et al., Methods in Molecular Biology 24:7-331, 1994. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and can be used with the present invention.

Unless otherwise stated, sequence identity/similarity values provided herein refer to the value obtained using the BLAST 2.0 suite of programs using default parameters. Altschul et al., Nucleic Acids Res, 2:3389-3402, 1997. It is to be understood that default settings of these parameters can be readily changed as needed in the future.

As those ordinary skilled in the art will understand, BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput Chem, 17:149-163, 1993) and XNU (Clayerie and States, Comput Chem, 17:191-1, 1993) low-complexity filters can be employed alone or in combination.

(c) “Sequence identity” or “identity” in the context of two nucleic acid or polypeptide sequences includes reference to the residues in the two sequences which are the same when aligned for maximum correspondence over a specified comparison window, and can take into consideration additions, deletions and substitutions. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have sequence similarity. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers and Miller, Computer Applic Biol Sci, 4: 11-17, 1988, for example, as implemented in the program PC/GENE (Intelligenetics, Mountain View, Calif., USA).

(d) “Percentage of sequence identity” means the value determined by comparing two optimally aligned sequences over a comparison window, wherein the portion of the polynucleotide sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e) (i) The term “substantial identity” or “homologous” in their various grammatical forms means that a polynucleotide comprises a sequence that has a desired identity, for example, at least 60% identity, preferably at least 70% sequence identity, more preferably at least 80%, still more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, for example, when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid, although such cross-reactivity is not required for two polypeptides to be deemed substantially identical.

(e) (ii) The terms “substantial identity” or “homologous” in their various grammatical forms in the context of a peptide indicates that a peptide comprises a sequence that has a desired identity, for example, at least 60% identity, preferably at least 70% sequence identity to a reference sequence, more preferably 80%, still more preferably 85%, most preferably at least 90% or 95% sequence identity to the reference sequence over a specified comparison window. Preferably, optimal alignment is conducted using the homology alignment algorithm of Needleman and Wunsch, J Mol Biol, 48:443, 1970. An indication that two peptide sequences are substantially identical is that one peptide is immunologically reactive with antibodies raised against the second peptide, although such cross-reactivity is not required for two polypeptides to be deemed substantially identical. Thus, a peptide is substantially identical to a second peptide, for example, where the two peptides differ only by a conservative substitution. Peptides which are “substantially similar” share sequences as noted above except that residue positions which are not identical may differ by conservative amino acid changes. Conservative substitutions typically include, but are not limited to, substitutions within the following groups: glycine and alanine; valine, isoleucine, and leucine; aspartic acid and glutamic acid; asparagine and glutamine; serine and threonine; lysine and arginine; and phenylalanine and tyrosine.

“Inhibitors,” “activators,” “modulators,” and “regulators” refer to molecules that activate, inhibit, modulate, regulate and/or block an identified function. For example, referring to enzymatic function of CODH, such molecules may be identified using in vitro and in vivo assays of CODH. Inhibitors are compounds that partially or totally block CODH activity, decrease, prevent, or delay its activation, or desensitize its cellular response. This may be accomplished by binding to CODH proteins directly or via other intermediate molecules. An antagonist or an antibody that blocks CODH activity, including inhibition of enzymatic function of CODH, is considered to be such an inhibitor. Activators are compounds that bind to CODH protein directly or via other intermediate molecules, thereby increasing or enhancing its activity, stimulating or accelerating its activation, or sensitizing its cellular response. An agonist of CODH is considered to be such an activator. A modulator can be an inhibitor or activator. A modulator may or may not bind CODH or its protein directly; it affects or changes the activity or activation of CODH or the cellular sensitivity to CODH. A modulator also may be a compound, for example, a small molecule, that inhibits transcription of CODH mRNA. In a desirable embodiment, an inhibitor of CODH is provided together with a substrate or competitive inhibitor of CODH, to facilitate formation of an active form of CODH (bound to substrate or competitive inhibitor) that may be more readily recognized by an antibody or antibody binding fragment such as a monoclonal Fab.

The group of inhibitors, activators and modulators of this invention also includes genetically modified versions of CODH, for example, versions with altered activity. The group thus is inclusive of the naturally occurring protein as well as synthetic ligands, antagonists, agonists, antibodies, small chemical molecules and the like.

“Assays for inhibitors, activators, or modulators” refer to experimental procedures including, for example, expressing CODH in vitro, in cells, applying putative inhibitor, activator, or modulator compounds, and then determining the functional effects on CODH activity, as described above. Samples that contain or are suspected of containing CODH are treated with a potential activator, inhibitor, or modulator. The extent of activation, inhibition, or change is examined by comparing the activity measurement from the samples of interest to control samples. A threshold level is established to assess activation or inhibition. For example, inhibition of a CODH polypeptide is considered achieved when the CODH activity value relative to the control is 80% or lower. Similarly, activation of a CODH polypeptide is considered achieved when the CODH activity value relative to the control is two or more fold higher.

An “isolated nucleic acid molecule” can refer to a nucleic acid molecule, depending upon the circumstance, that is separated from the 5′ and 3′ coding sequences of genes or gene fragments contiguous in the naturally occurring genome of an organism. The term “isolated nucleic acid molecule” also includes nucleic acid molecules which are not naturally occurring, for example, nucleic acid molecules created by recombinant DNA techniques.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral methyl phosphonates, 2-O-methyl ribonucleotides, and peptide-nucleic acids (PNAs).

Unless otherwise indicated, a particular nucleic acid sequence also implicitly encompasses conservatively modified variants thereof (for example, degenerate codon substitutions) and complementary sequences, as well as the sequence explicitly indicated. Specifically, degenerate codon substitutions may be achieved by generating sequences in which the third position of one or more selected (or all) codons is substituted with suitable mixed base and/or deoxyinosine residues (Batzer et al., Nucleic Acid Res, 19:081, 1991; Ohtsuka et al., J Biol Chem, 260:2600-2608, 1985; Rossolini et al., Mol Cell Probes, 8:91-98, 1994). The term nucleic acid is used interchangeably with gene, cDNA, mRNA, oligonucleotide, and polynucleotide.

A “host cell” is a naturally occurring cell or a transformed cell that contains an expression vector and supports the replication or expression of the expression vector. Host cells may be cultured cells, explants, cells in vivo, and the like. Host cells may be prokaryotic cells, for example, E. coli, or eukaryotic cells, for example, yeast, insect, amphibian, or mammalian cells, for example, CHO, HeLa, and the like.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, for example, hydroxyproline, -carboxyglutamate, and O-phosphoserine, phosphothreonine. “Amino acid analogs” refer to compounds that have the same basic chemical structure as a naturally occurring amino acid, i.e., a carbon that is bound to a hydrogen, a carboxyl group, an amino group, and an R group, for example, homoserine, norleucine, methionine sulfoxide, methionine methyl sulfonium. Such analogs have modified R groups (for example, norleucine) or modified peptide backbones, but retain the same basic chemical structure as a naturally occurring amino acid. “Amino acid mimetics” refers to chemical compounds that have a structure that is different from the general chemical structure of an amino acid, but that function in a manner similar to a naturally occurring amino acid. Amino acids and analogs are well known in the art.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” apply to both amino acid and nucleic acid sequences. With respect to particular nucleic acid sequences, conservatively modified variants refers to those nucleic acids which encode identical or similar amino acid sequences and include degenerate sequences. For example, the codons GCA, GCC, GCG and GCU all encode alanine. Thus, at every amino acid position where an alanine is specified, any of these codons can be used interchangeably in constructing a corresponding nucleotide sequence. The resulting nucleic acid variants are conservatively modified variants, since they encode the same protein (assuming that is the only alternation in the sequence). One skilled in the art recognizes that each codon in a nucleic acid, except for AUG (sole codon for methionine) and UGG (tryptophan), can be modified conservatively to yield a functionally-identical peptide or protein molecule. As to amino acid sequences, one skilled in the art will recognize that substitutions, deletions, or additions to a polypeptide or protein sequence which alter, add or delete a single amino acid or a small number (typically less than ten) of amino acids is a “conservatively modified variant” where the alteration results in the substitution of an amino acid with a chemically similar amino acid. Conservative substitutions are well known in the art and include, for example, the changes of: alanine to serine; arginine to lysine; asparigine to glutamine or histidine; aspartate to glutamate; cysteine to serine; glutamine to asparigine; glutamate to aspartate; glycine to proline; histidine to asparigine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine.

The terms “protein”, “peptide” and “polypeptide” are used herein to describe any chain of amino acids, regardless of length or post-translational modification (for example, glycosylation or phosphorylation). Thus, the terms can be used interchangeably herein to refer to a polymer of amino acid residues. The terms also apply to amino acid polymers in which one or more amino acid residue is an artificial chemical mimetic of a corresponding naturally occurring amino acid. Thus, the term “polypeptide” includes full-length, naturally occurring proteins as well as recombinantly or synthetically produced polypeptides that correspond to a full-length naturally occurring protein or to particular domains or portions of a naturally occurring protein. The term also encompasses mature proteins which have an added amino-terminal methionine to facilitate expression in prokaryotic cells.

The polypeptides of the invention can be chemically synthesized or synthesized by recombinant DNA methods; or, they can be purified from tissues in which they are naturally expressed, according to standard biochemical methods of purification. Also included in the invention are “functional polypeptides,” which possess one or more of the biological functions or activities of a protein or polypeptide of the invention. These functions or activities include the ability to bind some or all of the proteins which normally bind to CODH protein.

The functional polypeptides may contain a primary amino acid sequence that has been modified from that considered to be the standard sequence of CODH protein described herein. Preferably these modifications are conservative amino acid substitutions, as described herein.

A “label” or a “detectable moiety” is a composition that when linked with the nucleic acid or protein molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. A “labeled nucleic acid or oligonucleotide probe” is one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe may be detected by detecting the presence of the label bound to the nucleic acid or probe.

As used herein a “nucleic acid or oligonucleotide probe” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. It will be understood by one of skill in the art that probes may bind target sequences lacking complete complementarity with the probe sequence depending upon the stringency of the hybridization conditions. The probes are preferably directly labeled with isotopes, for example, chromophores, lumiphores, chromogens, or indirectly labeled with biotin to which a streptavidin complex may later bind. By assaying for the presence or absence of the probe, one can detect the presence or absence of a target gene of interest.

The phrase “selectively (or specifically) hybridizes to” refers to the binding, duplexing, or hybridizing of a molecule only to a particular nucleotide sequence under stringent hybridization conditions when that sequence is present in a complex mixture (for example, total cellular or library DNA or RNA).

The phrase “stringent hybridization conditions” refers to conditions under which a probe will hybridize to its target complementary sequence, typically in a complex mixture of nucleic acids, but to no other sequences. Stringent conditions are sequence-dependent and circumstance-dependent; for example, longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). In the context of the present invention, as used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other.

Generally, stringent conditions are selected to be about 5-10.degree. C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30.degree. C. for short probes (for example, 10 to 50 nucleotides) and at least about 60.degree. C. for long probes (for example, greater than 50 nucleotides). Stringent conditions may also be achieved with the addition of destabilizing agents, for example, formamide. For selective or specific hybridization, a positive signal is at least two times background, preferably 10 times background hybridization.

Exemplary, non-limiting stringent hybridization conditions can be as following: 50% formamide, 5.times.SSC, and 1% SDS, incubating at 42.degree. C., or, 5.times.SSC, 1 SDS, incubating at 65.degree. C., with wash in 0.2.times.SSC, and 0.1% SDS at 65.degree. C. Alternative conditions include, for example, conditions at least as stringent as hybridization at 68.degree. C. for 20 hours, followed by washing in 2.times.SSC, 0.1% SDS, twice for 30 minutes at 55.degree. C. and three times for 15 minutes at 60.degree. C. Another alternative set of conditions is hybridization in 6.times.SSC at about 45.degree. C., followed by one or more washes in 0.2.times.SSC, 0.1% SDS at 50-65.degree. C. For PCR, a temperature of about 36.degree. C. is typical for low stringency amplification, although annealing temperatures may vary between about 32.degree. C. and 48.degree. C. depending on primer length. For high stringency PCR amplification, a temperature of about 62.degree. C. is typical, although high stringency annealing temperatures can range from about 50.degree. C. to about 65.degree. C., depending on the primer length and specificity. Typical cycle conditions for both high and low stringency amplifications include a denaturation phase of 90.degree. C.-95.degree. C. for 30 sec.-2 min., an annealing phase lasting 30 sec.-2 min., and an extension phase of about 72.degree. C. for 1-2 min.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37.degree. C., and a wash in 1.times.SSC at 45.degree. C. A positive hybridization is at least twice background. Those of ordinary skill will readily recognize that alternative hybridization and wash conditions can be utilized to provide conditions of similar stringency.

“Antibody” refers to a polypeptide comprising a framework region encoded by an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 2 kDa) and one “heavy” chain (up to about 70 kDa). Antibodies exist, for example, as intact immunoglobulins or as a number of well-characterized fragments produced by digestion with various peptidases. While various antibody fragments are defined in terms of the digestion of an intact antibody, one of skill in the art will appreciate that such fragments may be synthesized de novo chemically or via recombinant DNA methodologies. Thus, the term antibody, as used herein, also includes antibody fragments produced by the modification of whole antibodies, those synthesized de novo using recombinant DNA methodologies (for example, single chain Fv), humanized antibodies, and those identified using phage display libraries (see, for example, Knappik et al., J Mol Biol, 296:57-86, 2000; McCafferty et al., Nature, 348:2-4, 1990), for example. For preparation of antibodies—recombinant, monoclonal, or polyclonal antibodies—any technique known in the art can be used in this invention (see, for example, Kohler & Milstein, Nature, 26:49-497, 1997; Kozbor et al., Immunology Today, 4:72, 1983; Cole et al., pp. 77-96 in Monoclonal Antibodies and Cancer Therapy, Alan R. Liss, Inc., 1998).

Techniques for the production of single chain antibodies (See U.S. Pat. No. 4,946,778) can be adapted to produce antibodies to polypeptides of this invention. Transgenic mice, or other organisms, for example, other mammals, may be used to express humanized antibodies. Phage display technology can also be used to identify antibodies and heteromeric Fab fragments that specifically bind to selected antigens (see, for example, McCafferty et al., Nature, 348:2-4, 1990; Marks et al., Biotechnology,:779-783, 1992).

“Blocking antibody” is a type of antibody, as described above, refers to polypeptides comprising a framework region encoded by an immunoglobulin gene or fragments thereof that specifically bind and block biological activities of an antigen, for example, a blocking antibody to CODH blocks the enzymatic function of CODH gene. A blocking antibody binds to a critical regions of a polypeptide and thereby inhibits its function. Critical regions include protein-protein interactions sites, such as active sites, functional domains, ligand binding sites, and recognition sites. A blocking antibody differs from other antibodies that it has an important role in blocking the function of the marker protein (for example, Erbitux). This distinguishes blocking antibodies from ones that kill a tumor cell just by virtue of the marker being present on the cell-surface, which involves no functional role (for example, Rituxan).

An “anti-CODH” antibody is an antibody or antibody fragment that specifically binds a polypeptide encoded by a CODH gene, cDNA, or a subsequence thereof. Anti-CODH antibody also includes a blocking antibody that inhibits enzymatic function of CODH.

Active substance homologs with modified specificity can be synthesized readily by substituting the non-cysteine residues of the conserved pentapeptide. Such recombinant mutants are well known in the art; substitution of the four as with other natural and synthetic as is readily performed. Activity is tested by the methods disclosed in the cited references.

Embodiments of the present invention utilize purified Enzyme polypeptides from natural or recombinant sources, or cells transformed with recombinant nucleic acid molecules encoding Enzyme. Various methods for the isolation of the Enzyme polypeptides may be accomplished by procedures well known in the art. For example, such polypeptides may be purified by immunoaffinity chromatography by employing the antibodies provided by the present invention. Various other methods of protein purification well known in the art include those described in Deutscher M (1990) Methods in Enzymology, Vol 182, Academic Press, San Diego Calif.; and Scopes R (1982) Protein Purification: Principles and Practice. Springer-Verlag, New York City, both incorporated herein by reference.

Use of Nucleotide Sequences for Methods and Compositions of the Invention

The nucleotide sequences encoding Enzyme (or their complement) have numerous applications in techniques known to those skilled in the art of molecular biology. These techniques include use as hybridization probes, use in the construction of oligomers for PCR, use for chromosome and gene mapping, use in the recombinant production of Enzyme, and use in generation of anti-sense DNA or RNA, their chemical analogs and the like. Uses of nucleotides encoding Enzyme disclosed herein are exemplary of known techniques and are not intended to limit their use in any technique known to a person of ordinary skill in the art. Furthermore, the nucleotide sequences disclosed herein may be used in molecular biology techniques that have not yet been developed, provided the new techniques rely on properties of nucleotide sequences that are currently known, e.g., the triplet genetic code, specific base pair interactions, etc.

It will be appreciated by those skilled in the art that as a result of the degeneracy of the genetic code, a multitude of Enzyme-encoding nucleotide sequences, some bearing minimal homology to the nucleotide sequence of any known and naturally occurring gene may be produced. The invention has specifically contemplated each and every possible variation of nucleotide sequence that could be made by selecting combinations based on possible codon choices. These combinations are made in accordance with the standard triplet genetic code as applied to the nucleotide sequence of naturally occurring Enzyme, and all such variations are to be considered as being specifically disclosed.

Although the nucleotide sequences which encode Enzyme and/or its variants are preferably capable of hybridizing to the nucleotide sequence of the naturally occurring Enzyme under stringent conditions, it may be advantageous to produce nucleotide sequences encoding Enzyme or its derivatives possessing a substantially different codon usage. Codons can be selected to increase the rate at which expression of the peptide occurs in a particular prokaryotic or eukaryotic expression host in accordance with the frequency with which particular codons are utilized by the host. Other reasons for substantially altering the nucleotide sequence encoding Enzyme and/or its derivatives without altering the encoded as sequence include the production of RNA transcripts having more desirable properties, such as a greater half-life, than transcripts produced from the naturally occurring sequence.

Nucleotide sequences encoding Enzyme may be joined to a variety of other nucleotide sequences by means of well established recombinant DNA, techniques (cf Sambrook J et al., supra). Useful nucleotide sequences for joining to Enzyme include an assortment of cloning vectors, e.g., plasmids, cosmids, lambda phage derivatives, phagemids, and the like, that are well known in the art. Vectors of interest include expression vectors, replication vectors, probe generation vectors, sequencing vectors, and the like. In general, vectors of interest may contain an origin of replication functional in at least one organism, convenient restriction endonuclease sensitive sites, and selectable markers for the host cell.

Another aspect of the subject invention is to provide for Enzyme-specific nucleic acid hybridization probes capable of hybridizing with naturally occurring nucleotide sequences encoding Enzyme. Such probes may also be used for the detection of similar Enzyme encoding sequences and should preferably contain at least 50% of the nucleotides from the conserved region or active site. The hybridization probes of the subject invention may be derived from the nucleotide sequences known for the Enzymes or from genomic sequences including promoters, enhancer elements and/or possible introns of the respective naturally occurring Enzymes. Hybridization probes may be labeled by a variety of reporter groups, including radionuclides such as .sup.32 P or .sup.35 S, or enzymatic labels such as alkaline phosphatase coupled to the probe via avidin/biotin coupling systems, and the like.

PCR as described U.S. Pat. Nos. 4,683,195; 4,800,195; and 4,965,188 provides additional uses for oligonucleotides based upon the nucleotide sequence which encodes Enzyme. Such probes used in PCR may be of recombinant origin, may be chemically synthesized, or a mixture of both and comprise a discrete nucleotide sequence for diagnostic use or a degenerate pool of possible sequences for identification of closely related genomic sequences.

Other means of producing specific hybridization probes for Enzyme DNAs include the cloning of nucleic acid sequences encoding Enzyme or Enzyme derivatives into vectors for the production of mRNA probes. Such vectors are known in the art and are commercially available and may be used to synthesize RNA probes in vitro by means of the addition of the appropriate RNA polymerase as T7 or SP6 RNA polymerase and the appropriate radioactively labeled nucleotides.

It is now possible to produce a DNA sequence, or portions thereof, encoding Enzyme and their derivatives entirely by synthetic chemistry, after which the gene can be inserted into any of the many available DNA vectors using reagents, vectors and cells that are known in the art at the time of the filing of this application. Moreover, synthetic chemistry may be used to introduce mutations into the Enzyme sequences or any portion thereof.

The nucleotide sequence can be used in an assay to detect inflammation or disease associated with abnormal levels of expression of Enzyme. The nucleotide sequence can be labeled by methods known in the art and added to a fluid or tissue sample from a patient under hybridizing conditions. After an incubation period, the sample is washed with a compatible fluid which optionally contains a dye (or other label requiring a developer) if the nucleotide has been labeled with an enzyme. After the compatible fluid is rinsed off, the dye is quantified and compared with a standard. If the amount of dye is significantly elevated, the nucleotide sequence has hybridized with the sample, and the assay indicates the presence of inflammation and/or disease.

The nucleotide sequence for Enzyme can be used to construct hybridization probes for mapping that gene. The nucleotide sequence provided herein may be mapped to a particular chromosome or to specific regions of that chromosome using well known genetic and/or chromosomal mapping techniques. These techniques include in situ hybridization, linkage analysis against known chromosomal markers, hybridization screening with libraries, flow-sorted chromosomal preparations, or artificial chromosome constructions YAC, P1 or BAC constructions. The technique of fluorescent in situ hybridization of chromosome spreads has been described, among other places, in Verma et al (1988) Human Chromosomes: A Manual of Basic Techniques, Pergamon Press, New York City.

Fluorescent in situ hybridization of chromosomal preparations and other physical chromosome mapping techniques may be correlated with additional genetic map data. Examples of genetic map data can be found in the 1994 Genome Issue of Science (265:1981 f). Correlation between the location of Enzyme on a physical chromosomal map and a specific disease (or predisposition to a specific disease) can help delimit the region of DNA associated with that genetic disease. The nucleotide sequence of the subject invention may be used to detect differences in gene sequence between normal and carrier or affected individuals.

Nucleotide sequences encoding Enzyme may be used to produce purified Enzyme using well known methods of recombinant DNA technology. Among the many publications that teach methods for the expression of genes after they have been isolated is Goeddel (1990) Gene Expression Technology, Methods and Enzymology, Vol 185, Academic Press, San Diego Calif. Enzyme may be expressed in a variety of host cells, either prokaryotic or eukaryotic. Host cells may be from the same species in which Enzyme nucleotide sequences are endogenous or from a different species. Advantages of producing Enzyme by recombinant DNA technology include obtaining adequate amounts of the protein for purification and the availability of simplified purification procedures.

Cells transformed with DNA encoding Enzyme may be cultured under conditions suitable for the expression of Enzyme and recovery of the protein from the cell culture. Enzyme produced by a recombinant cell may be secreted or may be contained intracellularly, depending on the Enzyme sequence and the genetic construction used. In general, it is more convenient to prepare recombinant proteins in secreted form. Purification steps vary with the production process and the particular protein produced.

In addition to recombinant production, fragments of Enzyme may be produced by direct peptide synthesis using solid-phase techniques (cf Stewart et al (1969) Solid-Phase Peptide Synthesis, W E Freeman Co, San Francisco Calif.; Merrifield J (1963) J Am Chem Soc 85:2149-2154. In vitro protein synthesis may be performed using manual techniques or by automation. Automated synthesis may be achieved, for example, using Applied Biosystems 431A Peptide Synthesizer (Foster City, Calif. Calif.) in accordance with the instructions provided by the manufacturer. Various fragments of Enzyme may be chemically synthesized separately and combined using chemical methods to produce the full length molecule.

Enzyme for antibody induction does not require biological activity; however, the protein must be immunogenic. Peptides used to induce specific antibodies may have an as sequence consisting of at least five aa, preferably at least 10 aa. They should mimic a portion of the as sequence of the protein and may contain the entire as sequence of a small naturally occurring molecule such as Enzyme. Short stretches of Enzyme as may be fused with those of another protein such as keyhole limpet hemocyanin and the chimeric molecule used for antibody production.

Antibodies specific for Enzyme may be produced by inoculation of an appropriate animal with the polypeptide or an antigenic fragment. An antibody is specific for Enzyme if it is produced against an epitope of the polypeptide and binds to at least part of the natural or recombinant protein. Antibody production includes not only the stimulation of an immune response by injection into animals, but also analogous steps in the production of synthetic antibodies or other specific-binding molecules such as the screening of recombinant immunoglobulin libraries (cf Orlandi R et al (1989) PNAS 86:3833-3837; Huse W D et al (1989) Science 256:1275-1281) or the in vitro stimulation of lymphocyte populations. Current technology (Winter G and Milstein C (1991) Nature 349:293-299) provides for a number of highly specific binding reagents based on the principles of antibody formation. These techniques may be adapted to produce molecules specifically binding Enzymes.

An additional embodiment of the subject invention is the use of Enzyme specific antibodies, inhibitors, receptors or their analogs as bioactive agents to treat activated monocyte disorders, such as inflammatory bowel disease, insulin-dependent diabetes mellitus, rheumatoid arthritis, septic shock and similar pathologic problems.

Bioactive compositions comprising agonists, antagonists, receptors or inhibitors of Enzyme may be administered in a suitable therapeutic dose determined by any of several methodologies including clinical studies on mammalian species to determine maximal tolerable dose and on normal human subjects to determine safe dose. Additionally, the bioactive agent may be complexed with a variety of well established compounds or compositions which enhance stability or pharmacological properties such as half-life. It is contemplated that the therapeutic, bioactive composition may be delivered by intravenous infusion into the bloodstream or any other effective means which could be used for treating problems involving Enzyme production and function.

An antisense strand coding for an Enzyme can be used either in vitro or in vivo to inhibit expression of the protein. Such technology is now well known in the art, and probes can be designed at various locations along the nucleotide sequence. By treatment of cells or whole test animals with such antisense sequences, the gene of interest can effectively be turned off. Frequently, the function of the gene can be ascertained by observing behavior at the cellular, tissue or organismal level (e.g. lethality, loss of differentiated function, changes in morphology, etc.).

In addition to using sequences constructed to interrupt transcription of the open reading frame, modifications of gene expression can be obtained by designing antisense sequences to intron regions, promoter/enhancer elements, or even to trans-acting regulatory genes. Similarly, inhibition can be achieved using Hogeboom base-pairing methodology, also known as “triple helix” base pairing.

Expression of Enzyme

Expression of Enzyme may be accomplished by subcloning the cDNAs into appropriate expression vectors and transfecting the vectors into appropriate expression hosts. In this particular case, the cloning vector previously used for the generation of the tissue library also provide for direct expression of the included Enzyme sequence in E. coli. Upstream of the cloning site, this vector contains a promoter for .beta.-galactosidase, followed by sequence containing the amino terminal Met and the subsequent 7 residues of .beta.-galactosidase. Immediately following these eight residues is an engineered bacteriophage promoter useful for artificial priming and transcription and a number of unique restriction sites, including Eco RI, for cloning.

Induction of the isolated, transfected bacterial strain with IPTG using standard methods will produce a fusion protein corresponding to the first seven residues of beta galactosidase, about 15 residues of “linker”, and the peptide encoded within the cDNA. Since cDNA clone inserts are generated by an essentially random process, there is one chance in three that the included cDNA will lie in the correct frame for proper translation. If the cDNA is not in the proper reading frame, it can be obtained by deletion or insertion of the appropriate number of bases by well known methods including in vitro mutagenesis, digestion with exonuclease III or mung bean nuclease, or oligonucleotide linker inclusion.

The Enzyme cDNA can be shuttled into other vectors known to be useful for expression of protein in specific hosts. Oligonucleotide amplimers containing cloning sites as well as a segment of DNA sufficient to hybridize to stretches at both ends of the target cDNA (25 bases) can be synthesized chemically by standard methods. These primers can then used to amplify the desired gene segments by PCR. The resulting new gene segments can be digested with appropriate restriction enzymes under standard conditions and isolated by gel electrophoresis. Alternately, similar gene segments can be produced by digestion of the cDNA with appropriate restriction enzymes and filling in the missing gene segments with chemically synthesized oligonucleotides. Segments of the coding sequence from more than one gene can be ligated together and cloned in appropriate vectors to optimize expression of recombinant sequence.

Suitable expression hosts for such chimeric molecules include but are not limited to mammalian cells such as Chinese Hamster Ovary (CHO) and human 293 cells, insect cells such as Sf9 cells, yeast cells such as Saccharomyces cerevisiae, and bacteria such as E. coli. For each of these cell systems, a useful expression vector may also include an origin of replication to allow propagation in bacteria and a selectable marker such as the .beta.lactamase antibiotic resistance gene to allow selection in bacteria. In addition, the vectors may include a second selectable marker such as the neomycin phosphotransferase gene to allow selection in transfected eukaryotic host cells. Vectors for use in eukaryotic expression hosts may require RNA processing elements such as 3′ polyadenylation sequences if such are not part of the cDNA of interest.

Additionally, the vector may contain promoters or enhancers which increase gene expression. Such promoters are host specific and include MMTV, SV40, or metallothionine promoters for CHO cells; trp, lac, tac or T7 promoters for bacterial hosts; or alpha factor, alcohol oxidase or PGH promoters for yeast. Transcription enhancers, such as the rous sarcoma virus (RSV) enhancer, may be used in mammalian host cells. Once homogeneous cultures of recombinant cells are obtained through standard culture methods, large quantities of recombinantly produced Enzyme can be recovered from the conditioned medium and analyzed using chromatographic methods known in the art. Because expression of the Enzyme protein may be lethal to certain cell types, care should be given to the selection of a suitable host species. Alternately, the protein can be expressed in the inactive form, such as in inclusion bodies. The inclusion bodies can be separated from the cells, the protein solubilized and refolded into active form.

Isolation of Recombinant Enzyme

Enzyme may be expressed as a chimeric protein with one or more additional polypeptide domains added to facilitate protein purification. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle Wash.). The inclusion of a cleavable linker sequence such as Factor XA or enterokinase (Invitrogen, San Diego Calif.) between the purification domain and the Enzyme sequence may be useful to facilitate expression of Enzyme.

Production of Enzyme Specific Antibodies

Two approaches are utilized to raise antibodies to Enzyme, and each approach is useful for generating either polyclonal or monoclonal antibodies. In one approach, denatured protein from the reverse phase HPLC separation is obtained in quantities up to 75 mg. This denatured protein can be used to immunize mice or rabbits using standard protocols; about 100 micrograms are adequate for immunization of a mouse, while up to 1 mg might be used to immunize a rabbit. For identifying mouse hybridomas, the denatured protein can be radioiodinated and used to screen potential murine B-cell hybridomas for those which produce antibody. This procedure requires only small quantities of protein, such that 20 mg would be sufficient for labeling and screening of several thousand clones.

In the second approach, the amino acid sequence of Enzyme, as deduced from translation of the cDNA, is analyzed to determine regions of high immunogenicity. Oligopeptides comprising appropriate hydrophilic regions, as shown in FIG. 3, are synthesized and used in suitable immunization protocols to raise antibodies. Analysis to select appropriate epitopes is described by Ausubel F M et al (supra). The optimal amino acid sequences for immunization are usually at the C-terminus, the N-terminus and those intervening, hydrophilic regions of the polypeptide which are likely to be exposed to the external environment when the protein is in its natural conformation.

Typically, selected peptides, about residues in length, are synthesized using an Applied Biosystems Peptide Synthesizer Model 431 A using finoc-chemistry and coupled to keyhole limpet hemocyanin (KLH, Sigma) by reaction with M-maleimidobenzoyl-N-hydroxysuccinimide ester (MBS; cf. Ausubel F M et al, supra). If necessary, a cysteine may be introduced at the N-terminus of the peptide to permit coupling to KLH. Rabbits are immunized with the peptide-KLH complex in complete Freund's adjuvant. The resulting antisera are tested for antipeptide activity by binding the peptide to plastic, blocking with 1% BSA, reacting with antisera, washing and reacting with labeled (radioactive or fluorescent), affinity purified, specific goat anti-rabbit IgG.

Hybridomas may also be prepared and screened using standard techniques. Hybridomas of interest are detected by screening with labeled Enzyme to identify those fusions producing the monoclonal antibody with the desired specificity. In a typical protocol, wells of plates (FAST; Becton-Dickinson, Palo Alto, Calif.) are coated with affinity purified, specific rabbit-anti-mouse antibodies (or suitable anti-species Ig) at 10 mglmi. The coated wells are blocked with 1% BSA, washed and exposed to supernatants from hybridomas. After incubation the wells are exposed to labeled Enzyme, 1 mg/ml. Clones producing antibodies will bind a quantity of labeled Enzyme which is detectable above background. Such clones are expanded and subjected to 2 cycles of cloning at limiting dilution (1 cell/3 wells). Cloned hybridomas are injected into pristine nice to produce ascites, and monoclonal antibody is purified from mouse ascitic fluid by affinity chromatography on Protein A. Monoclonal antibodies with affinities of at least 10e8 Me-1, preferably 10e9 to 10e10 or stronger, will typically be made by standard procedures as described in Harlow and Lane (1988) Antibodies: A Laboratory Manual. Cold Spring Harbor Laboratory, Cold Spring Harbor N.Y.; and in Goding (1986) Monoclonal Antibodies: Principles and Practice, Academic Press, New York City, both incorporated herein by reference.

Diagnostic Test Using EMme Specific Antibodies

Particular Enzyme antibodies are useful for the diagnosis of prepathologic conditions, and chronic or acute diseases which are characterized by differences in the amount or distribution of Enzyme. To date, Enzyme has been found only in the activated THP-1 library and is thus associated with abnormalities or pathologies which activate monocytes.

Diagnostic tests for Enzyme include methods utilizing the antibody and a label to detect Enzyme in human body fluids, tissues or extracts of such tissues. The polypeptides and antibodies of the present invention may be used with or without modification. Frequently, the polypeptides and antibodies will be labeled by joining them, either covalently or noncovalently, with a substance which provides for a detectable signal. A wide variety of labels and conjugation techniques are known and have been reported extensively in both the scientific and patent literature. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced as shown in U.S. Pat. No. 4,816,567, incorporated herein by reference.

A variety of protocols for measuring soluble or membrane-bound Enzyme, using either polyclonal or monoclonal antibodies specific for the respective protein are known in the art. Examples include enzyme-linked immunosorbent assay (ELISA), radioimmunoassay (RIA) and fluorescent activated cell sorting (FACS). A two-site monoclonal-based immunoassay utilizing monoclonal antibodies reactive to two non-interfering epitopes on Enzyme is preferred, but a competitive binding assay may be employed. These assays are described, among other places, in Maddox, D E et al (1983, J Exp Med 158:1211).

Purification of Native Enzyme Using Specific Antibodies

Native or recombinant Enzyme can be purified by immunoaffinity chromatography using antibodies specific for Enzyme. In general, an immunoaffinity column is constructed by covalently coupling the anti-Enzyme antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated Sepharose (Pharmacia LKB Biotechnology). The antibody is coupled to the resin, the resin is blocked, and the derivative resin is washed according to the manufacturer's instructions.

Such immunoaffinity columns are utilized in the purification of Enzyme by preparing a fraction from cells containing Enzyme in a soluble form. This preparation is derived by solubilization of the whole cell or of a subcellular fraction obtained via differential centrifugation by the addition of detergent or by other methods well known in the art. Alternatively, soluble Enzyme containing a signal sequence may be secreted in useful quantity into the medium in which the cells are grown.

A soluble Enzyme-containing preparation is passed over the immunoaffinity column, and the column is washed under conditions that allow the preferential absorbance of Enzyme (e.g., high ionic strength buffers in the presence of detergent). Then, the column is eluted under conditions that disrupt antibody/Enzyme binding (e.g., a buffer of pH 2-3 or a high concentration of a chaotrope such as urea or thiocyanate ion), and Enzyme is collected.

Use of an Enzyme in Drug Screening to Create a Microbe Inhibiting Age

Embodiments are particularly useful for screening compounds by using Enzyme polypeptide or binding fragments thereof in any of a variety of drug screening techniques. The Enzyme polypeptide or fragment employed in such a test may either be free in solution, affixed to a solid support, borne on a cell surface or located intracellularly. One method of drug screening utilizes eukaryotic or prokaryotic host cells which are stably transformed with recombinant nucleic acids expressing the polypeptide or fragment. Drugs are screened against such transformed cells in competitive binding assays. Such cells, either in viable or fixed form, can be used for standard binding assays. One may measure, for example, the formation of complexes between Enzyme and the agent being tested. Alternatively, one can examine the diminution in complex formation between Enzyme and its target cell, the monocyte or macrophage, caused by the agent being tested.

Thus, embodiments provide methods of screening for drugs, natural inhibitors or any other agents which can affect disease. These methods comprise contacting such an agent with a Enzyme polypeptide or fragment thereof and assaying 1) for the presence of a complex between the agent and the Enzyme polypeptide or fragment, or 2) for the presence of a complex between the Enzyme polypeptide or fragment and the cell, by methods well known in the art. In such competitive binding assays, the Enzyme polypeptide or fragment is typically labeled. After suitable incubation, free Enzyme polypeptide or fragment is separated from that present in bound form, and the amount of free or uncomplexed label is a measure of the ability of the particular agent to bind to Enzyme or to interfere with the Enzyme and agent complex.

Another technique for drug screening provides high throughput screening for compounds having suitable binding affinity to the Enzyme polypeptide and is described in detail in European Patent Application 84103564, published on Sep. 13, 1984, incorporated herein by reference. Briefly stated, large numbers of different small peptide test compounds are synthesized on a solid substrate, such as plastic pins or some other surface. The peptide test compounds are reacted with Enzyme polypeptide and washed. Bound Enzyme polypeptide is then detected by methods well known in the art. Purified Enzyme can also be coated directly onto plates for use in the aforementioned drug screening techniques. In addition, non-neutralizing antibodies can be used to capture the peptide and immobilize it on the solid support.

Competitive drug screening assays may be used in which neutralizing antibodies capable of binding Enzyme specifically compete with a test compound for binding to Enzyme polypeptides or fragments thereof. In this manner, the antibodies can be used to detect the presence of any peptide which shares one or more antigenic determinants with Enzyme.

Use of a Microbe Inhibiting Agent for Therapy

Antibodies, inhibitors, or antagonists of Enzyme (Microbe Inhibiting Agents) can provide different effects when administered therapeutically. Microbe Inhibiting Agent will be formulated in a nontoxic, inert, pharmaceutically acceptable aqueous carrier medium preferably at a pH of about 5 to 8, more preferably 6 to 8, although the pH may vary according to the characteristics of the antibody, inhibitor, or antagonist being formulated and the condition to be treated. Characteristics of Microbe Inhibiting Agent include solubility of the molecule, half-life and immunogenicity; these and other characteristics may aid in defining an effective carrier. Native human proteins are preferred as Microbe Inhibiting Agents, but organic or synthetic molecules resulting from drug screens may be equally effective in particular situations.

Microbe Inhibiting Agents may be delivered by known routes of administration including but not limited to topical creams and gels; transmucosal spray and aerosol, transdermal patch and bandage; injectable; intravenous and lavage formulations; and orally administered liquids and pills, particularly formulated to resist stomach acid and enzymes. The particular formulation, exact dosage, and route of administration will be determined by the attending physician and will vary according to each specific situation.

Such determinations are made by considering multiple variables such as the condition to be treated, the Microbe Inhibiting Agent to be administered, and the pharmacokinetic profile of the particular Microbe Inhibiting Agent. Additional factors which may be taken into account include disease state (e.g. severity) of the patient, age, weight, gender, diet, time of administration, drug combination, reaction sensitivities, and tolerance/response to therapy. Long-acting Microbe Inhibiting Agent formulations might be administered only once per day, or even less often: every 3 to 4 days, every week, or every two weeks depending on half-life and clearance rate of the particular Microbe Inhibiting Agent.

Normal dosage amounts may vary from 0.1 to 100,000 micrograms, up to a total dose of about 1 g, depending upon the route of administration. Guidance as to particular dosages and methods of delivery is provided in the literature; see U.S. Pat. No. 4,657,760; 5,206,344; or 5,225,212. It is anticipated that different formulations will be effective for different Microbe Inhibiting Agent and that administration targeting the eosinophil may necessitate delivery in a manner different from that to another organ or tissue. It is contemplated that other infectious diseases such as tubuculosis may also be treatable with a Microbe Inhibiting Agent.

CODH Antigen as a Microbe Inhibiting Agent

Any composition that contains one or more epitopes of CODH is useful to stimulate antibodies against microbe for practice of the invention. Particularly preferred are CODH proteins, peptide fragments and DNA encoding at least part of the CODH and which can be administered to produce an immune response.

Anti-CODH Antibodies as Microbe Inhibitiniz Agents

Any composition that contains at least one anti-CODH antibody, antibody fragment or other antibody binding site specific to CODH is suitable, particularly as a therapeutic for alleviating and/or curing one or more disease states mentioned herein.

Microbe Inhibiting Agents Derived or Designed from other M. TB Proteins

The inventor discovered CODH in the genome of M. tuberculosis. Thus, monoclonal and polyclonal antibodies directed against CODH in the diagnosis treatment and prevention of tuberculosis may be used for disease that is undetectable by conventional screening methods in this, or any, immune-suppressed population.

Although small changes in sequence may exist in CODH found in TB versus other microorganisms in which the enzyme occurs, the enzyme theoretically would be disarmed the same way by a method or material in accordance with the present invention. An important step in the development of materials and method in accordance with the invention is to obtain serum from some tubercular patients and perform gel electrophoresis to purify the CODH found in those patients and to sequence it. Further, the same procedure may be repeated for M. pneumonia.

Nucleic Acid as Microbe Inhibiting Agents

DNA vaccines can be made using sequence information from the genome of M. tuberculosis and such sequences readily are available to the skilled artisan. Most preferably a DNA vaccine will contain sequence information from the CODH gene. Likewise, an antisense composition may be used to turn down or turn off synthesis of one or more 5 enzymes needed by the TB organism or one of the other organisms described herein.

Preferred Enzyme Targets of Microbe Inhibiting Agents

In designing a Microbe Inhibiting Agent, it is preferred to pick an Enzyme according to the invention. The Enzyme, or portions of it, may then be used to formulate a suitable antigen inhibitor, antibody, and the like as described herein. Some classes of Microbe Inhibiting Agents are briefly reviewed next.

(i) Inhibitors of Carbon Monoxide Dehydrogenase (CODH).

Methods for the large-scale purification of the CO dehydrogenase from Methanosarcina thermophila (see Example 1 below) allow high throughput screening of inhibitors to identify potential antibiotics. The large-scale purification also allows for determination of the crystal structure which, with the primary sequence deduced from the genes encoding the CO dehydrogenase, will lead to location of the active site and identification of residues essential for catalysis providing a foundation for rational drug design.

(ii) Chelators of Carbon Monoxide Dehydrogenase (CODH)

Prior to 1992, the functions of the clusters in CODH were not really known. Shin et al. (Shin, et al., J. Am. Chem. Soc. 114:9718-9719, 1992) disclosed that the Nickel (Ni) ion in one of the clusters (subsequently named the A-cluster) could be selectively removed by adding a metal chelating agent (1,10phenanthroline or “phen”). Without this Ni, the enzyme can't catalyze the synthesis of acetyl-coenzyme A. The chelator phen has two N donor atoms arranged so they chelate metal ions using two cis sites; that's why the Ni is probably labile (see FIG. 1). Ni was then implicated in one or more reactions of CODH.

Ni can be removed by chelation and such chelators are specifically contemplated as antibiotics for practice of the embodiments of the invention. An example of a nickel chelator is hexahistidine. In one embodiment, hexahistidine is added to the diet of a patient in a pharmaceutically effective amount. Preferably the hexahistidine is added with an excipient at more than 10 ug/100 g of wet food for at least one month. In another embodiment the hexahistidine is covalently conjugated to a non-digestible substance, such as a water swellable polymer and added to the food in that form. The hexahistidine preferably is excreted by the body, with attached nickel. Thus, the hexahistidine works by chelating nickel and preventing use of nickel by microorganisms. In another embodiment the nickel chelator is a histidine containing compound obtained from a nickel accumulating Alyssum species of the Brassica plant family.

Molybdenum on the other had also is a part of CODH structural cluster and can be removed by using chelating agents. Non-toxic Ni and Mo chelatots can thus be used to inactivate CODH. For example, a list of non-toxic Ni and Mo chelators are shown in Table 1, which can be used to inactivate or inhibit CODH activities.

1TABLE 1 Non-toxic Ni & Mo chelators Chelator Chelating Action Ref. Calcium ethylene diamine Mo: Acute psychosis Arh Hig Rada tetraacetic acid (CaEDTA) symptoms remitted several Toksikol September 1999; hours after the start of 50(3):289-97 chelation therapy with calcium ethylene diamine tetraacetic acid (CaEDTA). Ni-specific chelator, Ni: Ni in the CO(L)-deficient Biochemistry July 2000 dimethylglyoxime. CODH can be removed by 11; 39(27):7956-63 treatment. 10% Ni: Demonstrated the Contact Dermatitis diethylenetriaminepentaacetic preventive effect of 10% April 2001; 44(4):224-8 acid (DTPA) in an oil-in-water diethylenetriaminepentaacetic emulsion. acid (DTPA) in an oil-in-water emulsion in nickel-sensitized patients 1. Tetraethylthiuram Ni: These chelators increased Pharmacol Toxicol disulphide (disulfiram, the whole-body retention of November 1994; 75(5):285-Antabuse, TTD). nickel also when given by 93 2. Sodium intraperitoneal injection diethyldithiocarbamate (DDC). shortly after oral or intraperitoneal administration of nickel. Cyclam, a known specific Ni: The pretreatment of Biochem Int chelator of nickel. cyclam, a known specific 1990; 20(3):495-501 chelator of nickel restored free radical reductase and glutathione 5-transferase activities and alleviated nickel mediated enhancement of lipid peroxidation. Meso-2,3-dimercaptosuccinic Metal ion: Meso-2,3-Altem Med Rev acid (DMSA). dimercaptosuccinic acid June 1998; 3(3): 199-207 (DMSA) is a sulihydryl-containing, water-soluble, non-toxic, orally-administered metal chelator which has been in use as an antidote to heavy metal toxicity since the 1950s.

Design of New Microbe Inhibiting Agents vie Rational Drug Design from 3-Dimensional Information of an Enzyme

In important embodiment is potentiation of new treatment modalities and substances based on inhibition of an Enzyme used by TP. Another embodiment is potentiation of new treatment modalities and substances based on inhibition of the CODH and related Enzymes used by other organisms such as the TB. Accordingly, the inventor specifically intends that the 3-dimensional structures of such Enzymes be obtained and used for rational drug design of inhibitors of the Enzymes.

The 3-d information is used by an acceptable procedure for drug design and in fact, the 3-D information itself is a valuable tool that allows a drug company to derive an important pharmaceutical simply by possession of the 3-dimensional structural information of the enzyme. This is particularly helpful when the 3-dimensional structure is of a transition state of the enzyme, because this particular structure shows the type of inhibitor that best interferes with Enzyme function.

The power of rational drug design was reviewed by Bugg et al., Drugs by Design, 92 Scientific American in December, 1993 (also see N. Cohen, “Rational Drug Design and Molecular Modeling”, Drugs of the Future, 10, pp. 311-328 (1985). A requirement of rational drug design is the production of crystals of the desired target protein which provide for the determination of the detailed atomic structure of both the parent protein and its complex with the pharmaceutical.

One procedure useful in structure-based rational drug design is docking (reviewed in Blaney, J. M. and Dixon, J. S., Perspectives in Drug Discovery and Design, 1993, 1, 301). Docking provides a means for using computational tools and available structural data on macromolecules to obtain new information about binding sites and molecular interactions. Docking is the placement of a putative ligand in an appropriate configuration for interacting with a receptor. Docking can be accomplished by geometric matching of a ligand and its receptor, or by minimizing the energy of interaction. Geometric matching is faster and can be based on descriptors or on fragments.

Structure-based drug design efforts often encounter difficulties in obtaining the crystal structure of the target and predicting the binding modes for new compounds. The difficulties in translating the structural information gained from X-ray crystallography into WO 00178342 PCT/US00/16679 a useful guide for drug synthesis calls for continued effort in the development of computational tools. Qualitative assessments of RT-inhibitor complexes provide helpful information, and advances in the field supersede the earlier art summarized in the above references, making systematic quantitative prediction of inhibitory activity of new 5 compounds based on structural information of a reality.

Embodiments of the invention disclosed herein addresses the need for a structure by providing a model for the three-dimensional structure of an enzyme used by TP.

Of course, the goal of rational drug design is to produce structural analogs of biologically active polypeptides of interest or of small molecules with which they interact, e.g., agonists, antagonists, or inhibitors. Any of these examples can be used to fashion drugs which are more active or stable forms of the polypeptide or which enhance or interfere with the function of a polypeptide in vivo (cf Hodgson J., Bio/Technology, 9:19-21, 1991, incorporated herein by reference).

In one approach reviewed above, the three-dimensional structure of a protein of interest, or of a protein-inhibitor complex, is determined by x-ray crystallography, by computer modeling or, most typically, by a combination of the two approaches. Both the shape and charges of the polypeptide must be ascertained to elucidate the structure and to determine active site(s) of the molecule. Less often, useful information regarding the structure of a polypeptide may be gained by modeling based on the structure of homologous proteins. However, the crystal structure of one active substance protein is known (Walker, 1994, supra) and can be used as a starting point. In both cases, relevant structural information is used to design analogous Enzyme-like molecules or to identify efficient inhibitors. Useful examples of rational drug design include molecules which have different specificity or improved activity or stability as shown by Braxton S and Wells J A, Biochemistry, 31:7796-7801, 1992) or which act as inhibitors, agonists, or antagonists of native peptides as shown by Athauda et al., J Biochem, 113:742-746, 1993), incorporated herein by reference.

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of embodiments of the present invention, sufficient amount of polypeptide may be made available to perform such analytical studies as X-ray crystallography. In addition, knowledge of the Enzyme amino acid sequence provided herein will provide guidance to those employing computer modeling techniques in place of or in addition to x-ray crystallography.

Use of a Microbe Inhibiting Agent as a Medicine

Generally, a Microbe Inhibiting Agent of the present invention as described above will be administered in a pharmaceutical composition to an individual already showing signs of “AIDS” or other disease described herein or at high risk of such infection. Those in the incubation phase or the acute phase of infection can be treated with the immunogenic, immunoactive or antibiotic substance separately or in conjunction with other treatments, as appropriate. In therapeutic applications, compositions are administered to a patient in an amount sufficient to elicit an effective B cell and/or T cell response to microbe or to hinder or kill the microbe and to cure or at least partially arrest its symptoms and/or complications. An amount adequate to accomplish this is defined as a “therapeutically or prophylactically effective dose” which may be an “immune response provoking amount” or a “lethal dose amount.” Amounts effective for a therapeutic or prophylactic use will depend on, e.g., the stage and severity of the disease the age, weight, and general state of health of the patient, and the judgment of the prescribing physician. The size of the dose will also be determined by the active substance composition, method of administration, timing and frequency of administration as well as the existence, nature, and extent of any adverse side-effects that might accompany the administration of a particular compound(s) and the desired physiological effect. It will be appreciated by one of skill in the art that various conditions or disease states may require prolonged treatment involving multiple administrations.

Suitable doses and dosage regimens can be determined by conventional range-finding techniques known to those of ordinary skill in the art. Generally, treatment is initiated with smaller dosages that are less than the optimum dose of the compound. Thereafter, the dosage is increased by small increments until the optimum effect under the circumstances is reached. The present inventive method typically will involve the administration of about 0.1 mg to about 50 mg of one or more of the compounds described above per kg body weight of the individual. For a 70 kg-patient, dosages of from about 10 mg to about 100 mg of active substance would be more commonly used, followed by booster dosages from about 0.01 mg to about 1 mg of active substance over weeks to months, depending on a patient's immune response.

It must be kept in mind that the active substances and compositions of the present invention may generally be employed in serious disease states, that is, life-threatening or potentially life threatening situations. In such cases, in view of the minimization of extraneous substances and the relative nontoxic nature of the active substances, it is possible and may be felt desirable by the treating physician to administer substantial excesses of these active substance compositions.

Single or multiple administrations of the compositions can be carried out with dose levels and pattern being selected by the treating physician. In any event, the pharmaceutical formulations should provide a quantity of B cell and/or T cell stimulatory active substances of the invention sufficient to effectively treat the patient. For therapeutic use, administration should begin at the first sign of microbe infection or shortly after diagnosis in cases of acute infection, and continue until at least symptoms are substantially abated and for a period thereafter. In well-established and chronic cases, loading doses followed by maintenance or booster doses may be required.

The pharmaceutical compositions for therapeutic treatment are intended for parenteral, topical, oral or local administration and generally comprise a pharmaceutically acceptable carrier and an amount of the active ingredient sufficient to reverse or prevent the bad effects of microbe infection. The carrier may be any of those conventionally used and is limited only by chemico-physical considerations, such as solubility and lack of reactivity with the compound, and by the route of administration.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

The pharmaceutically acceptable excipients described herein, for example, vehicles, adjuvants, carriers or diluents, are well-known to those who are skilled in the art and are readily available to the public. It is preferred that the pharmaceutically acceptable carrier be one that is chemically inert to the active compounds and one that has no detrimental side effects or toxicity under the conditions of use. Such carriers can include immuno-stimulating complexes (i.e. cholesterol, saponin, phospholipid peptide complexes), aluminum hydroxide (alum), heat shock proteins, linkage to synthetic microspheres (polyamino-microspheres-).

The choice of excipient will be determined in part by the particular method used to administer the composition. Accordingly, there is a wide variety of suitable formulations of the pharmaceutical composition of the present invention.

The following formulations for oral, aerosol, parenteral, subcutaneous, intravenous, intramuscular, interperitoneal, rectal, and vaginal administration are merely exemplary and are in no way limiting. Preferably, the pharmaceutical compositions are administered parenterally, e.g., intravenously, subcutaneously, intradermally, or intramuscularly. Thus, the invention provides compositions for parenteral administration that comprise a solution of the stimulatory active substances dissolved or suspended in an acceptable carrier suitable for parenteral administration, including aqueous and non-aqueous, isotonic sterile injection solutions.

Overall, the requirements for effective pharmaceutical carriers for parenteral compositions are well known to those of ordinary skill in the art. See Pharmaceutics and Pharmacy Practice, J. B. Lippincott Company, Philadelphia, Pa., Banker and Chalmers, eds., pages 238-250, (1982), and ASHP Handbook on Injectable Drugs, Toissel, 4th ed., pages 622-630 (1986). Such solutions can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils useful in parenteral formulations include petroleum, animal, vegetable, or synthetic oils. Specific examples of oils useful in such formulations include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-beta-aminopropionates, and 2-alkyl-imidazoline quaternary ammonium salts, and (e) mixtures thereof.

The parenteral formulations typically will contain from about 0.5 to about 25% by weight of the active ingredient in solution. Preservatives and buffers may be used: In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Topical formulations, including those that are useful for transdermal drug release, are well-known to those of skill in the art and are suitable in the context of the present invention for application to skin.

Formulations suitable for oral administration require extra considerations considering the particular molecular nature of the Microbe Inhibiting Agent and the likely breakdown thereof if such compounds are administered orally without protecting them from the digestive secretions of the gastrointestinal tract. Such a formulation can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The Microbe Inhibiting Agent molecules of the present invention, alone or in combination with other suitable components, can be made into aerosol formulations to be administered via inhalation. For aerosol administration, the active substances are preferably supplied in finely divided form along with a surfactant and propellant. Typical percentages of the material are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Pulmonary administration of aerosol formulations in particular are desired and a wide variety of delivery techniques may be used. See U.S. No. 20070014736 which is incorporated specifically by reference in its entirety, and particularly the methods, devices and materials for pulmonary administration of materials such as protein. In particular, see the article “Dry Powder Inhalers” by Mahavir Chougule et al. in Recent Patents on Drug Delivery & Formulation 2007, vol. 1 pp. 11-20, the contents of which are incorporated by reference in their entirety. Desirably any of the therapeutic materials described herein may be delivered by an inhaler via a technique known to skilled artisans.

Mixed esters, such as mixed or natural glycerides may be employed. The surfactant may constitute 0.1%-20% by weight of the composition, preferably 0.25-5%. The balance of the composition is ordinarily propellant. A carrier can also be included as desired, e.g., lecithin for intranasal delivery. These aerosol formulations can be placed into acceptable pressurized propellants, such as dichlorodifluoromethane, propane, nitrogen, and the like. They also may be formulated as pharmaceuticals for non-pressured preparations, such as in a nebulizer or an atomizer. Such spray formulations may be used to spray mucosa.

Additionally, the compounds and polymers useful in the present inventive methods may be made into suppositories by mixing with a variety of bases, such as emulsifying bases or water-soluble bases. Formulations suitable for vaginal administration may be presented as peccaries, tampons, creams, gels, pastes, foams, or spray formulas containing, in addition to the active ingredient, such carriers as are known in the art to be appropriate.

In some embodiments particularly where epitopes of CODH are used to stimulate an immune response, it is desirable to include in the pharmaceutical composition at least one component that primes CTL generally. Lipids have been identified that are capable of priming CTL in vivo against viral antigens, e.g., tripalmitoyl-5-glycerylcysteinly-seryl-serine (P sub 3 CSS), which can effectively prime virus specific cytotoxic T lymphocytes when covalently attached to an appropriate immunoactive substance. See, Deres et al., Nature, 342:561-564, 1989. Active substances of the present invention can be coupled to P sub 3 CSS, for example and the ipoprotein administered to an individual to specifically prime a cytotoxic T lymphocyte response to microbe.

The concentration of the Microbe Inhibiting Agent of the present invention in the pharmaceutical formulations can vary widely, i.e., from less than about 1%, usually at or at least about 10% to as much as 20 to 50% or more by weight, and will be selected primarily by fluid volumes, viscosities, etc., in accordance with the particular mode of administration selected.

Thus, a typical pharmaceutical composition for intravenous infusion could be made up to contain 250 ml of sterile Ringer's solution, and 100 mg of active substance. Actual methods for preparing parenterally administrable compounds will be known or apparent to those skilled in the art and are described in more detail in, for example, Remington's Pharmaceutical Science (17th ed., Mack Publishing Company, Easton, Pa., 1985).

It will be appreciated by one of ordinary skill in the art that, in addition to the aforedescribed pharmaceutical compositions, the compounds of the present inventive method may be formulated as inclusion complexes, such as cyclodextrin inclusion complexes, or liposomes. Liposomes serve to target the compounds to a particular tissue, such as lymphoid tissue or microbe-infected cells. Liposomes can also be used to increase the half-life of the active substance composition. Liposomes useful in the present invention include emulsions, foams, micelles, insoluble monolayers, liquid crystals, phospholipid dispersions, lamellar layers and the like. In these preparations the active substance to be delivered is incorporated as part of a liposome, alone or in conjunction with a molecule which binds to, e.g., a receptor, prevalent among lymphoid cells, such as monoclonal antibodies which bind to the antigen, or with other therapeutic or immunogenic compositions. Thus, liposomes filled with a desired active substance of the invention can be directed to the site of infection, where the liposomes then deliver the selected therapeutic/immunogenic active substance compositions. Liposomes for use in the invention are formed from standard vesicle-forming lipids, which generally include neutral and negatively charged phospholipids and a sterol, such as cholesterol. The selection of lipids is generally guided by consideration of, for example, liposome size and stability of the liposomes in the blood stream.

A variety of methods are available for preparing liposomes, as described in, for example, Szoka et al., Ann. Rev. Biophys. Bioeng., 9, 467 (1980), and U.S. Pat. Nos. 4,235,871, 4,501,728, 4,837,028 and 5,019,369. For targeting to the immune cells, a ligand to be incorporated into the liposome can include, for example, antibodies or fragments thereof specific for cell surface determinants of the desired immune system cells. A liposome suspension containing an active substance may be administered intravenously, locally, topically, etc. in a dose that varies according to the mode of administration, the active substance being delivered, the stage of disease being treated, etc.

In another aspect the invention is directed to vaccines that contain in addition to the Microbe Inhibiting Agent (having desired epitopes) as an active ingredient an immunogenically effective amount of a cytotoxic T-lymphocyte stimulating active substance having a sequence as described herein. Other immunomodulators may be added such as interleukin-1, beta (IL-1 beta) peptide and interleukin 12 (IL-12) peptide. Active substances may be for example, complexed to cholera toxin B subunit to stimulate mucosal immunity. The active substance(s) may be introduced into a patient linked to its own carrier or as a homopolymer or heteropolymer of active units. Such a polymer has the advantage of increased immunological reaction and, where different peptides are used to make up the polymer, the additional ability to induce antibodies and/or cytotoxic T cells that react with different antigenic determinants of microbe. Useful carriers are well known in the art, and include, e.g., keyhole limpet hemocyanin, thyroglobulin, albumins such as human serum albumin, tetanus toxoid, polyamino acids such as poly (D-lysine: D-glutamic acid), and the like. The vaccines can also contain a physiologically tolerable (acceptable) diluent such as water, phosphate buffered saline, or saline, and further typically include an adjuvant. Adjuvants such as incomplete Freund's adjuvant, aluminum phosphate, aluminum hydroxide, or alum or materials well known in the art. And, as mentioned above, cytotoxic T lymphocyte responses can be primed by conjugating active substances of the invention to lipids, such as P sub 3 CSS. Upon immunization with an active substance composition as described herein, via injection, aerosol, oral, transdermal or other route, the immune system of the host responds to the vaccine by producing large amounts of cytotoxic T-lymphocytes specific for microbe antigen, and the host becomes at least partially immune to microbe infection, or resistant to developing chronic microbe infection.

Vaccine compositions containing the active substances of the invention are administered to a patient susceptible to or otherwise at risk of microbe infection to enhance the patient's own immune response capabilities. Such an amount is defined to be a “immunogenically effective dose” or a “prophylactically effective dose.” In this use, the precise amounts again depend on the patient's state of health and weight, the mode of administration, the nature of the formulation, etc., but generally range from about 1.0 mg to about 500 mg per 70 kilogram patient, more commonly from about 50 mg to about 200 mg per 70 kg of body weight.

For therapeutic or immunization purposes when using a protein or nucleic acid as the Microbe Inhibiting Agent, the active substances of the invention can also be expressed by attenuated viral hosts, such as vaccinia. This approach involves the use of vaccinia virus as a vector to express nucleotide sequences that encode at least part of an Enzyme. Upon introduction into an microbe-infected host or into a non-infected host, the recombinant vaccinia virus expresses the Microbe Inhibiting Agent and thereby elicits a host cytotoxic T lymphocyte response to microbe. Vaccinia vectors and methods useful in immunization protocols are described in, e.g., U.S. Pat. No. 4,722,848. Another vector is BCG (bacille Calmette Guerin). BCG vectors are described in Stover et al., Nature, 351, 456-460 (1991). A wide variety of other vectors useful for therapeutic administration or immunization of the active substances of the invention, e.g., Salmonella typhi vectors and the like, will be apparent to those skilled in the art from the description herein.

The compositions and methods of the claimed invention may be employed for ex vivo therapy, wherein, as described briefly above, a portion of a patient's lymphocytes are removed, challenged with a stimulating dose of a active substance of the present invention, and the resultant stimulated cells are returned to the patient. Accordingly, in more detail, ex vivo therapy as used herein concerns the therapeutic or immunogenic manipulations that are performed outside the body on lymphocytes or other target cells that have been removed from a patient. Such cells are then cultured in vitro with high doses of the subject active substances, providing a stimulatory concentration of active substance in the cell medium far in excess of levels that could be accomplished or tolerated by the patient. Following treatment to stimulate the Cells the cells are returned to the host, thereby treating the microbe infection. The host's cells also may be exposed to vectors that carry genes encoding the active substances, as described above. Once transfected with the vectors, the cells may be propagated in vitro or returned to the patient. The cells that are propagated in vitro may be returned to the patient after reaching a predetermined cell density.

Certain disadvantages of conventional vaccines are overcome by using what is called “genetic immunization” (Tang, 1992). This technology involves inoculating simple, naked plasmid DNA encoding a pathogen active substance into the cells of the host. The pathogen's antigens in this case an Enzyme such as CODH, are produced intracellularly and, depending on the attached targeting signals, can be directed toward major histocompatibility complex (MHC) class I or II presentation. Risk of infection is essentially eliminated and the DNA can be delivered to cells not normally infected by the pathogen. Compared to conventional vaccines, the production of genetic vaccines is straightforward and DNA is considerably more stable than proteinaceous or live/attenuated vaccines. Genetic immunization (a.k.a. DNA, polynucleotide etc. immunization) with specific genes has shown promise in several model systems of pathogenic disease, and a few natural systems. Use of DNA (or RNA) thus overcomes some of the problems encountered when an animal is presented directly with an antigen.

Genetic immunization concerns DNA segments, that can be isolated from virtually any non-mammalian pathogen source, that are free from total genomic DNA and that encode the active substances disclosed herein. In addition these DNA segments may be synthesized entirely in vitro using methods that are well-known to those of skill in the art. As used herein, the term “DNA segment” refers to a DNA molecule that has been isolated free of total genomic DNA of a particular host species. Therefore, a DNA segment encoding a peptide or protein having a desired sequence refers to a DNA segment that contains these coding sequences yet is isolated away from, or purified free from, total genomic DNA of the species from which the DNA segment has been cloned. Included within the term “DNA segment,” are DNA segments and smaller fragments of such segments, and also recombinant vectors, including, for example, plasmids, cosmids, phagemids, phage, viruses, and the like.

Similarly, a DNA segment contemplated here refers to a DNA segment which may include in addition to peptide encoding sequences, certain other elements such as, regulatory sequences, isolated substantially away from other naturally occurring genes or peptide-encoding sequences. In this respect, the term “gene” is used for simplicity to refer to a functional protein, polypeptide or peptide-encoding unit. As will be understood by those in the art, this functional term includes both genomic sequences, cDNA sequences and smaller engineered gene segments that express, or may be adapted to express proteins, polypeptides or peptides.

“Isolated substantially away from other coding sequences” means that the gene of interest, in this case, a gene encoding microbe epitopes forms the significant part of the coding region of the DNA segment, and that the DNA segment does not contain large portions of naturally-occurring coding DNA, such as large chromosomal fragments or other functional genes or cDNA coding regions. Of course, this refers to the DNA segment as originally isolated, and does not exclude genes or coding regions later added to the segment by the hand of man.

The invention has broad implications that provide new substances (“Microbe Inhibiting Agents”) and methods of their generation and use. These Agents are made from known materials and methods for stimulating formation of antibodies that react with components of TB and/or other Enzymes, found in other anaerobes and facultative anaerobes. The Agents may also stimulate the formation of an immune response by nucleic acid, or may directly inhibit one or more Enzymes to inhibit a microbe. Accordingly, the invention encompasses and utilizes known prior art methods relating to each of these factors and efforts and a skilled artisan in each respective field now can prepare such materials and methods of their use for destroying, inhibiting and detecting TB and syphilis independently or when they occur, sometimes undetected, in AIDS, as well as appreciate the formation of a new class of antibiotics. The invention specifically contemplates and includes such optimization and applications. Nevertheless, the inventor has, in addition to discovered new methods and materials for purifying, studying and utilizing the enzymes implicated in these diseases. A summary of some of their results useful for practice of the invention follows.

Copper chelators are used in a desirable embodiment. The methods and chelators described, for example, in U.S. No. 20050159489, published Jul. 21, 2005 and U.S. Pat. No. 6,610,693 and naming John Baker are desirable for embodiments and are incorporated by reference in their entireties. Also desirable and incorporated by reference are U.S. Pat. No. 6,951,890 (Cooper et al) and U.S. Pat. No. 6,897,243. Examples of suitable copper-chelating compounds include triethylenetetramine dihydrochloride (triene), penicillamine, sar, diamsar, ethylenediamine tetraacetic acid, o-phenanthroline, histidine, and (where appropriate as a salt such as, for example, a suitable calcium sodium salt to avoid hypocalcemia): trientine (triene), ethylenediaminetetraacetic acid (EDTA), diethylenetriaminetetraacetic acid (DPTA), 2,2,2tetramine tetrahydrochloride (TETA), 2,3,2tetramine tetrahydrochloride, D-penicillamine (DPA), 1,4,8,11tetraazacyclotretradecane (Cyclam), 5,7,7′,12,14,14′hexamethyl-1,4,8,11tetraazacyclotretradecane (Cyclam S), Sodium 2,3dimercaptopropane-1-sulfonate (DMPS), N-acetylpenicillamine (NAPA), D-Penicillamine (PA),’ Desferroxamine, 2,3-dimercaptopropanol (BAL), 2,3-dimercaptosuccinic acid (DMSA), trithiomolybdate, 3-7-Diazanonan-1,9-diamin (BE 6184), 1,4,8,11-tetraazacyclotetradecane-1,4,8,1′-tetraacetic acid, 1,4,8,11-tetraazabicyclo[6.6.2]hexadecane, 4,11-bis(N,N-diethylamidomethyl)-1,4,8,11-tetraazabicyclo[6.6.2]hexadecane, 4,11-bis(amidoethyl)-1,4,8,11-tetraazabicyclo[6.6.2]hexadecane, melatonin, clioquinol, cuprizone, N,N′-diethyldithiocarbamate, zinc acetate, zinc salts, bathocuproinedisulfonic acid, bathocuprinedisulfonate, neocuproine (2,9-dimethyl-1,10-phenanthroline), tetrathiomolybdate, trimetazidine, triethylene tetramine tetrahydrochloride, 2,3,2-tetraamine, pyridine-2,6-bis(thiocarboxylic acid) or pyrrolidine dithiocarbamate, tetraethylenepentamine, N,N,N′,N-tetrakis(2-pyridylemethyl) ethylenediamine, 1,4,7,11-tetraazaundecane tetrahydrochloride, tetraethylenepentamine pentahydrochloride, D-Penicillamine (DPA), 1,10-orthophenanthroline, 3,4-Dihydroxybenzoic acid, 2,2′-bicinchinonic acid, diamsar, 3,4′,5, trihydroxystilbene (resveratrol), mercaptodextran, o-phenanthroline, disulfiram (antabuse), sar, calcium trisodium diethylenetriaminepentaacetate (salt of cpd above), and methimazole (1-methyl-2-thiolimidazole). The mode of administration, amounts and forms of administration as taught in U.S. Pat. No. 6,610,693 particularly are desired in embodiments. Such specific passages from this patent specifically are incorporated by reference. Triethylenetetramine is preferred. Chelators and other agents such as antibodies and antibody binding fragments that bind CODH may be administered by inhalation, as aerosols as well as orally and by injection.

### EXAMPLE

Large-Scale Purification of CO Dehydrogenase/acetyl-CoA Synthase from Methanosarcina thermophila

Methanosarcina thermophila strain TM-1 is cultured on acetate in a 100 liter pH auxostat. The basal medium contains (in grams per liter, final concentration): NH.sub.4Cl, 1.44; K.sub.2HPO.sub.4, 1.13; KH.sub.2PO.sub.4, 1.13; NaCl, 0.45; MgSO.sub.4.2H.sub.2O, 0.09; CaCl 1/2.sub.2 1/4.2H1/2 20, 0.06; yeast extract (Difco Laboratories), 0.5; Trypticase (BBL Microbiology systems), 0.5; Fe(NH.sub.4).sub.2(SO.sub.4).-sub.2, 0.01; cysteine.HCl, 0.27; Na.sub.2S.9H₂O, 0.27; Antifoam C, 0.5; and resazurin, 0.001. Trace elements and vitamin solutions are each added at a final concentration of 1% (vol/vol); NiC₁₋₆H.sub.2O is added to a final concentration of 0.5 g/liter. Sodium acetate (50 mM) is added as the substrate. When cells are cultured in the presence of NiCl.sub.2.6H.sub.2O trypticase is omitted, yeast extract is decreased to 0.1 g/liter, and Ni metal dissolved in nitric acid) is added to a final concentration of 0.5 mM. Cells are harvested in a continuous-flow centrifuge (Ceps type LE) under a stream of N.sub.2, and the resulting cell paste is frozen and stored in liquid nitrogen. The general anaerobic procedures for the preparation of cell extracts and for enzyme assays are as follows. All containers and solutions used for anaerobic procedures are made O.sub.2-free by repeated vacuum degassing and replacement with O.sub.2-free gas (N.sub.2, H.sub.2, or CO). All gasses used are scrubbed free of trace amounts of O₂ by passage through reduced BASF catalyst R3-11 (Chemical Dynamics, South Plainfield, N.J.). Cell extracts are prepared anaerobically under a H.sub.2 atmosphere. Breakage buffer consists of 50 mM potassium N-tris(hydroxymethyl)methyl-2-aminoethanesulfonate buffer (TES)(pH7.0) containing 10 mM 2-mercaptoethanol, 10 mM MgCl.sub.2, 5% (vol/vol) glycerol, and 0.015 mg/ml of DNase I (Sigma, St. Louis, Mo.). All steps for enzyme purification are performed in a Coy anaerobic chamber (Coy Manufacturing Co., Ann Arbor, Mich.) unless otherwise noted. Buffer A contains 50 mM TES (pH 6.8), 10% (vol/vol) ethylene glycol, and 10 mM MgCl1/2 2. Buffer B and buffer C are identical to buffer A except 1.0 M KCl and 0.15 M KCl are added. Saturated ammonium sulfate solution in 50 mM TES (pH 6.8) and 10 mM MgCl 1/2 2 are added to 10 ml of cell extract to-a final concentration of 0.35 saturation. This mixture was incubated for 30 min on ice and then centrifuged at 41,000.times.g for 20 min in a DuPont Sorvall RC-5B centrifuge. The brown supernatant solution containing CO dehydrogenase activity is dialyzed against 1.5 l of buffer A without ethylene glycol. The remaining steps in the purification utilize a high resolution fast protein liquid chromatography (FPLC) system (Pharmacia, Piscataway, N.J.) equipped with a model GP-250 gradient programmer. A sample (10 ml) of the dialyzed enzyme solution are injected onto a Mono-Q HR 10110 ion exchange column (Pharmacia) previously equilibrated with Buffer A. A linear gradient from 0.0 to 0.5M KCl is applied at a flow rate of 2.0 ml/min. Two peaks of CO dehydrogenase activity elute. The second, larger peak is collected and injected again onto the Mono-Q HR 10110 column equilibrated with buffer A. The enzyme is concentrated 10-fold by batch elution with 0.4 M KCl. Aliquots (0.5 ml) of the concentrated protein solution are injected on a Superose-6 (Pharmacia) gel filtration column previously equilibrated with Buffer C. The column is developed at a flow rate of 0.4 ml/min. Purified CO dehydrogenase is collected and stored in liquid N.sub.2.

All publications and patent applications cited in this disclosure are specifically incorporated by reference in their entireties.

It is intended that the specification be considered as exemplary only, with the true scope and spirit of the invention being indicated by the following claims.

1. A method for the treatment of Mycobacterium in a human subject, comprising the steps of:
a) providing a pharmaceutical composition comprising at least an anti-CODH antibody or CODH cofactor chelator; and
b) administering the composition of a) to the patient.

2. The method of claim 1, wherein the CODH cofactor chelator is a copper chelator.

3. The method of claim 2, wherein the CODH cofactor chelator is triethylenetetramine.

4. The method of claim 1, wherein the anti-CODH antibody is an antibody binding fragment that binds to CODH.

5. The method of claim 1, wherein step b) is carried out by pulmonary inhalation.

6. The method of claim 5, wherein the pharmaceutical composition further comprises a substrate or competitive inhibitor of CODH.

7. The method of claim 1, wherein the Myocobacterium is Mycobacterium tuberculosis or Mycobacterium pneumonia.

8. The method of claim 1, wherein the pharmaceutical composition further comprises another microbial for the treatment of Mycobacterium in the human.

9. A method for the treatment or alleviation of HIV symptoms in a human subject, comprising the steps of:
a) testing a body sample of the human subject for HIV;
b) in response to a positive test result, providing a pharmaceutical composition comprising at least an anti-CODH antibody or CODH cofactor chelator; and
c) administering the composition of b) to the patient.

10. The method of claim 9, wherein the CODH cofactor chelator comprises a nickel or molybdenum chelator.

11. The method of claim 9, wherein the CODH cofactor chelator is a copper chelator.

12. The method of claim 9, wherein the CODH cofactor chelator is triethylenetetramine.

13. The method of claim 9, wherein step c) is carried out by pulmonary inhalation.

14. The method of claim 9, wherein the pharmaceutical composition further comprises another microbial for the treatment of Mycobacterium in the human.

15. The method of claim 9, wherein the pharmaceutical composition further comprises a substrate or competitive inhibitor of CODH.

16. A composition for treatment or alleviation of HIV symptoms in a human subject, comprising: a pharmaceutical composition comprising at least an anti-CODH antibody or CODH cofactor chelator in a pharmaceutically acceptable carrier.

17. The composition of claim 16, wherein the anti-CODH antibody comprises monoclonal antibody binding fragments.

18. The composition of claim 16, wherein the composition is in a pulmonary inhalation form.

19. The composition of claim 16, further comprising another microbial for the treatment of Mycobacterium.

20. The composition of claim 16, wherein the chelator is triethylenetetramine.
